SEQUENCE LISTING (4 sequences)

SEQ ID NO: 1 - protein, 17 aa, Rattus norvegicus
RRKASGPPVSELITKAV

SEQ ID NO: 2 - protein, 50 aa, Bos sp.
  Bos sp. partial sequence
SETAPAAPAAAPPAEKTPVKKKAAKKPAGARRKASGPPVSELITKAVAAS

SEQ ID NO: 3 - protein, 50 aa, Homo sapiens
  Histone H1 partial sequence
SETAPAAPAAPAPAEKTPIKKKARKAAGGAKRKASGPPVSELITKAVAAS

SEQ ID NO: 4 - protein, 50 aa, Rattus norvegicus; identical to SEQ ID NO: 3
  Histone H1 partial sequence

(19) United States
(12) Patent Application Publication
(10) Pub. No.: US 2004/0052780 A1
(43) Pub. Date: Mar. 18, 2004

(54) Immunosuppresants

(75) Inventors: Kazuhisa Ono, Higashihiroshima-shi (JP); Seiji Kawamoto, Higashihiroshima-shi (JP); Takeshi Goto, Tsukuba-shi (JP); Shuji Sato, Kawasaki-shi (JP); Sigeru Goto, Oita-gun (JP)

Correspondence Address: FITCH EVEN TABIN AND FLANNERY, 120 SOUTH LA SALLE STREET, SUITE 1600, CHICAGO, IL, 60603-3406, US

(73) Assignee: Hisamitsu Pharmaceutical Co., Inc., Tosu-shi (JP)

(21) Appl. No.: 10/378,089

(22) Filed: Mar. 4, 2003

(30) Foreign Application Priority Data
Sep. 5, 2002 (JP) P2002-260681

(51) Int. Cl. A61K 39/395

(52) U.S. Cl. 424/130.1

(57) ABSTRACT

There are provided immunosuppressants with few side effects that can be used to suppress rejection involving organ transplantation, and the immunosuppressants comprising antibodies against histone H1 which specifically recognize antigenic proteins may be used to suppress immunity in mammals for treatment or prevention of rejection involving organ transplantation.

### BACKGROUND OF THE INVENTION

1. Field of the Invention

This invention relates to immunosuppressants. More specifically, it relates to pharmaceutical agents which comprise antibodies against histone H1 and which are useful as immunosuppressants (especially transplant immunosuppressants).

2. Related Background Art

With the dramatic advances that have been achieved in surgical techniques for organ transplantation to date, the success of organ transplant surgery has come to depend on controlling post-surgical transplant rejection. Rejection occurs when the body recognizes the graft as a foreign substance, provoking a series of immune reactions to eliminate it. Clinical immunosuppressants which have been conventionally used to suppress such immune reactions include steroid agents (e.g., prednisolone), antimetabolites (e.g., azathioprine, mizoribine) and antibiotics (e.g., cyclosporin A, tacrolimus). The emergence of such pharmaceutical agents has already helped established kidney, heart and liver transplants as common practice in medicine, while lung, pancreas and ileum transplants are also becoming more widely performed as results improve (Konnichi no Ishoku [Modern Transplantation], Vol. 11, No. 1, 53, 1998).

However, such pharmaceutical agents have innegligible side effects, and steroid agents, for example, while having a wider range of action than other immunosuppressants, generally exhibit weaker immunosuppressing effects, and sometimes reduced efficacy or the emergence of resistance are often seen when administered for prolonged periods. Azathioprine exhibits an immunosuppressing effect by inhibiting nucleic acid synthesis, but its use increases the risk of infection by bacteria and the like, and occasionally causes bone marrow suppression (leukopenia, thrombocytopenia, anemia) and liver damage. Mizoribine offers the advantage of producing less bone marrow suppression and liver damage compared to azathioprine, but its immunosuppressing effect is less than satisfactory. Cyclosporin A and tacrolimus are pharmacetical agents having very potent immunosuppressing activity, and have contributed to successful results when used for organ transplantation. However, they have been found to produce serious central nervous system toxicity and nephrotoxicity, and must therefore be used with the utmost care (Konnichi no Ishoku, Vol. 11, No. 1, 37, 1998).

On the other hand, the liver has come to be recognized as a very unique organ in organ transplantation. Success has been achieved with orthotopic liver transplantation (OLT) in some allogenic animal combinations without administering immunosuppressants (Nature, Vol. 223, 427, 1969). Also, the fact that immunosuppressive state results in cases of hepatocyte damage occurring with hepatitis and other conditions suggests that the damaged liver or regenerated liver may produce immunosuppressive substances (Nature, Vol. 251, 655, 1974).

These findings have been followed by reports to date of several immunosuppressive proteins, some of which are provided below.

(L-Arginase)

L-arginase separated from hepatocytes is an immunosuppressive protein thought to be released into the blood when liver is damaged. Release of a large amount of L-arginase leads to digestion of L-arginine, producing L-ornithine and urea and creating an arginine deficiency in the body which is believed to suppress the active proliferation of lymphocytes (J. Immunol., Vol. 131, 2427, 1983 and Japanese Unexamined Patent Publication HEI No. 3-157399; JPA 3-157399). However, since this condition is improved by administration of arginine, there is doubt as to whether rejection can be suppressed over a prolonged period in organisms with different arginine supply rates among individual cells, or for example, liver with a higher level of arginine production. Furthermore, digestion of a large amount of arginine also produces a large amount of urea, creating the clinically problematic condition of azotemia; and extended administration has been implicated in causing renal failure (Medicina, Vol. 31, No. 11, 60, 1994).

(Very Low-Density Lipoprotein (VLDL))

It is thought that this substance is secreted from hepatocytes and finds its way into the serum. VLDL inhibits DNA synthesis in peripheral monocytes and suppresses its activation (J. Immunol. Vol. 119, 2129, 1977). However, prolonged administration of VLDL as a pharmaceutical agent produces chronic increase in serum values of VLDL as well as intermediate-density lipoprotein (IDL) and low-density lipoprotein (LDL) which are converted by lipoprotein lipase, and possibly leading to hyperlipidemia, triglyceridemia, diabetes, urinemia and arteriosclerosis (Medicina Vol. 31, No. 11, 179, 1994).

(α-Fetoprotein and Early Pregnancy Factor)

It is known that immunosuppressive proteins are secreted by the liver under special conditions. α-fetoprotein, one of the known liver tumor markers, also has immunosuppressing power and has been identified and examined in fetal mice (J. Exp. Med., Vol. 141, 269, 1975). Early pregnancy factor is a protein with immunosuppressing power that has been detected in the urine and blood of pregnant women at their early pregnancy (Nature, Vol. 278, 649, 1979), and its secretion has also been confirmed after hepatic lobectomy. These proteins are substances that suppress the proliferation of lymphocytes in a non-specific manner and that are thought to be associated with the excellent regenerative ability of the liver which is exhibited in cases of liver damage. However, this has not been sufficient as an explanation for immunotolerance with liver transplantation.

(Soluble Class I Antigen)

In clinical liver transplantation, a high level of donor-type soluble class I antigen is found in recipients immediately after transplantation. The substance was believed to induce immunotolerance, but adequate immunosuppression has not been achieved by its administration in vivo (Transplantation, Vol. 50, 678, 1990).

(Liver Suppressor Factor-1: LSF-1)

This is an approximately 40 kD immunosuppressive protein which has been obtained by periodic sampling and SDS-PAGE electrophoresis of the serum of a post-liver transplantation rat, and is predicted to have a very notable effect; however, the protein has not been identified (Transplant. Immunol., Vol. 3, 174, 1995), and further study is required before it can be utilized as a pharmaceutical agent.

(Indoleamine 2,3-Dioxygenase (IDO))

IDO is an immunosuppressive protein derived from organs other than the liver. It has been shown that IDO is expressed by placental syncytiotrophoblasts and that it suppresses rejection of the conceptus from allogenic murine parents during its growth (Science, Vol. 281, 1191, 1998). An attempt to use IDO as a transplant immunosuppressant has also been reported (WO/50901A1 pamphlet).

(Anti-MHC Antibodies)

Antibodies against donor-type MHC class I antigen and class II antigen have been found in the sera of post-liver transplantation rats (Saibo Kogaku [Cell Engineering], Vol. 12, No. 5, 1993; Rinsho Kagaku [Clinical Science], Vol. 26, No. 6, 1990; Seitai Bogyo [Biological Defense], Vol. 7, No. 1, 1990). Anti-class I antibodies are thought to have an immunosuppressing effect by forming complexes with the soluble class I antigen. Also, anti-class II antibodies are thought to have an immunosuppressing effect by masking class II antigen (composed of a 35 kD chain and a 28-29 kD chain) present in explant tissue.

In summary, the clinical immunosuppressants currently used are known to have such side effects as bone marrow suppression, liver damage, central nervous system toxicity and nephrotoxicity. Moreover, among the immunosuppressive proteins found post-liver transplants, none has been discovered that possesses clinically applicable activity.

The present inventors have noted the immunosuppressing activity in the sera of rat models of allogenic orthotopic liver transplantation and have determined that it is attributable to an immunoglobulin-like substance which is found immediately after the liver transplantation (Japanese Unexamined Patent Publication No. 2001-233900; JPA 2002-233900). The immunoglobulin-like substance specifically recognizes three types of rat spleen-derived proteins (molecular weight/isoelectric point: 73 kD/5.2, 34 kD/4.9, 31 kD/5.3), and it was therefore concluded to be distinct from the aforementioned anti-MHC antibodies.

### SUMMARY OF THE INVENTION

It is an object of this invention to provide immunosuppressants with few of the aforementioned side effects, and particularly to provide antigen-specific transplant immunosuppressants capable of specific suppression against rejection in organ transplants.

As a result of diligent research directed toward solving the problems described above, the present inventors have discovered that the serum of a post-liver transplantation rat possesses immunosuppressing activity. It has also been found that central to this immunosuppressing activity is the IgG component of the serum, and that the antibodies recognize specific antigens (73, 34, 31 kD) on autolymphocytes. The 31 kD protein was isolated from the antigens and sequenced, and a fragment identified from the partial sequence structure was subjected to BLAST analysis. As a result, histone H1 proteins (rat, bovine, human) were identified as proteins having high homology with the sequence. Polyclonal antibodies for the histone H1 protein were obtained and their immunosuppressing activities examined to confirm the immunosuppressing effects. Their rejection-suppressing activities were also confirmed in a rat heterotopic heart transplant test, which is an accepted organ transplant rejection model. In addition, it was also confirmed that anti-histone polyclonal antibodies are expressed in the serum of a post-liver transplantation rat. This invention has been completed on the basis of these findings.

In other words, the invention provides an immunosuppressant comprising an antibody against histone H1.

The antibody against histone H1 as the effective ingredient of the immunosuppressant is preferably an anti-histone H1 polyclonal antibody.

The histone is more preferably human-derived.

The immunosuppressant is most preferably applied for transplant immunosuppression.

The invention also provides a pharmaceutical composition for immunosuppression comprising a therapeutically effective amount of an antibody against histone H1, together with a pharmaceutically acceptable carrier.

The antibody against histone H1 in the aforementioned pharmaceutical composition is preferably an anti-histone H1 polyclonal antibody.

The histone in the aforementioned pharmaceutical composition is preferably human-derived.

The invention further provides a method for treating a mammal including human in need of immunosuppression, which comprises administering to the mammal, a therapeutically effective amount of an antibody against histone H1.

In the aforementioned treatment method, the immunosuppression is preferably transplant immunosuppression.

The antibody against histone H1 in the aforementioned treatment method is preferably an anti-histone H1 polyclonal antibody.

The histone in the aforementioned treatment method is preferably human-derived.

The invention still further provides a method for preventing transplant rejection in a mammalian recipient including human, which comprises administering to the mammalian recipient, a therapeutically effective amount of an antibody against histone H1.

The invention still further provides an antigenic protein isolated and purified from rat spleen cells, characterized in that

the protein has a molecular weight of 31 kD as determined by SDS-PAGE and an isoelectric point of 5.3 as determined by isoelectric electrophoresis, and includes the partial amino acid sequence represented by RRKASGPPVSELITKAV.

The invention still further provides an antigenic protein isolated and purified from the serum of a post allogenic orthotopic liver transplantation rat, characterized in that

the protein has a molecular weight of 37 kD as determined by SDS-PAGE and an isoelectric point of about 5.0 as determined by isoelectric electrophoresis, and is expressed in the rat serum at a later stage post-transplantation.

The invention still further provides a method for diagnosing liver transplant rejection in a mammalian recipient including human, the method comprising:

a step of measuring the level of serum immunoreactivity against an autoantigenic protein expressed in the post-transplantation serum, wherein the level of immunoreactivity is used as an index for the rejection.

The invention still further provides the aforementioned diagnosis method, wherein the level of immunoreactivity is the anti-histone H1 antibody titer.

The immunosuppressants of this invention exhibit excellent immunosuppressing action and therefore are not only useful as transplant immunosuppressants for preventing rejection in organ and tissue transplants (especially liver transplant), but are also promising as therapeutic agents for autoimmune diseases and inflammatory diseases such as allergic conditions.

### BRIEF DESCRIPTION OF THE DRAWINGS

FIG. 1 is a bar graph showing the results of measuring immunosuppressing activities of post-liver transplantation sera by MLR assay.

FIG. 2 is an image showing the results of analysis of post-liver transplantation sera by Western blotting.

FIG. 3 is a pair of graphs showing the results of measuring immunosuppressing activities of IgG fractions and native sera after liver transplantation, by MLR assay. FIG. 3A shows the results for the sera on the 14th day post-liver transplantation, and FIG. 3B shows the results for the sera on the 63rd day post-liver transplantation.

FIG. 4 is a pair of images showing the results of analyzing antigens recognized by IgG in post-liver transplantation sera, by SDS-PAGE and Western blotting. FIG. 4A shows the results with Coumassie staining and FIG. 4B shows the results with immunostaining.

FIG. 5 is a bar graph showing the results of measuring immunosuppressing activities by MLR assay, after immunodepleting post-liver transplantation sera.

FIG. 6 shows the sequence alignment for a partial sequence of an antigenic protein derived from rat spleen cells, against partial sequences of rat, bovine and human histone H1 proteins.

FIG. 7 is an image showing the results of analyzing antigenic proteins derived from rat spleen cells, by SDS-PAGE and Western blotting. FIG. 7A shows the results with Coumassie staining and FIG. 7B shows the results with immunostaining.

FIG. 8 is a graph showing the results of measuring immunosuppressing activities of the anti-histone H1 antibodies by MLR assay.

FIG. 9 is a bar graph showing the results of measuring anti-histone H1 antibody titers in post-liver transplantation sera.

### DETAILED DESCRIPTION OF THE PREFERRED EMBODIMENTS

The invention will now be described in greater detail.

With respect to the proteins of this invention, their expression may be induced by carrying out orthotopic liver transplantation between xenogenic rat species (for example, from DA rats to PVG rats) by, for example, the method of Kamada et al. (Surgery, Vol. 93, 64, 1983). Specifically, a liver from a DA rat is transplanted into a PVG rat. Blood may be sampled after the liver transplantation, the supernatant from centrifugation collected and serum containing the protein obtained therefrom.

The method of purifying the proteins from the serum may be, for example, according to the procedure of Oda et al. (B.B.R.C. Vol. 204, No. 3, 1131, 1994). Specifically, the serum is passed through a DEAE Sepharose column, and the adsorbed protein fraction is eluted. It is further eluted with a CNBr-activated Sepharose column for purification. The obtained sample is electrophoresed by SDS-PAGE and the protein bands are collected.

The serum of a post-liver transplantation rat of the invention (hereinafter referred to either as “rat post-liver transplantation serum” or simply as “post-liver transplantation serum”) exhibits immunosuppressing activity which can be confirmed by mixed lymphocyte reaction (MLR) assay using rat spleen lymphocytes (John E. C. et al., Current protocols in immunology, Vol. 1, John Wiley & Sons, Inc., Brooklyn, 1994). These results demonstrated that an immunosuppressing factor is present in the serum of a post-transplantation recipient PVG rat. In addition, a Protein G column or the like may be used to purify the IgG fraction from the serum, or to remove it to prepare an IgG-eliminated fraction. Analysis of such fractions and serum by MLR assay has indicated that central to the immunosuppressing activity is the IgG fraction.

The IgG fraction specifically recognizes one or more antigenic proteins from rat spleen cells. The antigenic proteins are proteins with molecular weight/isoelectric point values of 31 kD/5.3, 34 kD/4.9 and 73 kD/5.2 (Japanese Unexamined Patent Publication No. 2001-233900; JPA 2001-233900, mentioned above).

Of these antigenic proteins, the N-terminal sequence portion of -the isolated and purified 31 kD protein was analyzed according to a conventional method and a homology search was conducted for the obtained sequence by BLAST analysis, where it was found to have high homology with the inferred epitope portion of the known protein human histone H1. The partial sequence is RRKASGPPVSELITKAV, as set forth in SEQ. ID. NO: 1 in the Sequence Listing.

The anti-histone H1 antibodies according to the invention include polyclonal and monoclonal antibodies, and they are raised by conventional methods as antibodies against histone H1 or its antigenic fragments. The term “antibodies” as used throughout the present specification refers to antibodies against the aforementioned histone H1 antigens, as well as their antibody fragments or derivatives and their modified forms (for example, Fab, F(ab)′₂ fragment, chimeric antibodies or humanized antibodies). Such derivatives and modified forms are prepared from antibodies for the purpose of enhancing immunosuppressing activity, reducing antigenicity, alleviating side effects or improving stability in the blood. Examples of such modified forms are antibodies modified with polyethylene glycol or dextran, which are known to have a prolonged half-life in the blood.

Polyclonal antibodies are prepared by first mixing the histone H1 immunogen with an adjuvant, immunosensitizing a mammal and obtaining antiserum from the animal. The immunosensitizing method is not particularly limited so long as it is a conventional method capable of obtaining antiserum, and Freund's complete adjuvant is suitable as the adjuvant.

The mammals which may be used include rabbits, rats, goats and the like, but rabbits are most suitable when considering the problem of securing antiserum quantities and breeding conditions. When a rabbit, for example, is to be immunized, it may be done subcutaneously, peritoneally, intravenously, or intramuscularly. While there are no particular limitations on the inoculation interval or inoculation dose, an interval of 2 weeks with 2-10 inoculations is preferred. The inoculation dose may be at least 10 μg or more, and preferably 100 μg to 10 mg, of histone H1 per injection. On the 3rd to 10th day, and preferably on the 7th day, after the final immunization, the whole blood is taken to obtain the antiserum. The antiserum may be purified by any of various methods commonly used for antibody purification, and affinity chromatography is particularly suitable. The purified antibody is concentrated and then adjusted to a concentration necessary for assay of the immunosuppressing activity by a conventional method (for example, MLR assay as mentioned above).

Monoclonal antibodies may be prepared according to the procedure of Koehier Milstein et al. (Koehier Milstein, Nature 256, 495, 1975). Specifically, they may be produced from a fused cell line (hybridoma) created by fusing a spleen cell from the immunized animal to a myeloma cell from an animal of the same species. Mouse myeloma cells are preferred as myeloma cells to be used for cell fusion because of their high cell fusion efficiency and proliferation rate. The immunosensitizing method is the same as for preparation of polyclonal antibodies as described above, but mice are preferably used as the animal. The spleen cells are extracted after the final immunization. The immunized mouse spleen-derived lymphocytes are then fused to appropriate myeloma cells. A fusing agent such as Sendai virus or polyethylene glycol (PEG) may be used for the fusion. The fused cells are selectively grown in a suitable medium (for example, HAT medium). The proliferated cells are screened for production of the antibody of interest, and a preferred cell line (monoclonal antibody-producing hybridoma clone) is selected. The obtained monoclonal antibody is purified by a conventional method. The purified antibody is adjusted to a concentration necessary for assay of the immunosuppressing activity, also by a conventional method.

Anti-histone H1 antibodies according to the invention exhibit immunosuppressing activity and are therefore useful in treatment regimen for treatment or prevention of graft versus host (recipient) reaction (rejection) occurring with the transplants of tissues or organs such as heart, kidney, liver, bone marrow, skin and the like, as well as for treatment or prevention of autoimmune diseases. Specifically, the anti-histone H1 antibody according to the invention is administered to a subject (a mammal including human) as the effective ingredient of immunosuppressant, in the form of a pharmaceutical composition together with a pharmaceutically acceptable carrier, in the same manner as ordinary pharmaceutical agents. For example, it may be dissolved in physiological saline for injection or distilled water for injection, and administered by drip infusion, intravenous injection, subcutaneous injection or the like. If necessary, an appropriate buffering agent, a preservative and a stabilizer (human serum albumin, glucose, etc.) may be included. When an immunosuppressant of the invention is used for transplant immunosuppression, it is preferably administered by perfusion or local injection into the transplanted organ, or utilizing a balloon catheter. For suppression of rejection in a liver transplant, portal vein administration is particularly preferred. The dosage will differ depending on the condition, age, etc. of the subject, but in most cases it may be administered at 0.1-100 mg/kg and preferably 1-10 mg/kg, either once or over several times. An anti-histone H1 antibody of the invention will be included in a pharmaceutical composition in an amount sufficient to exhibit the desired therapeutic effect, depending on the progression and state of the disease requiring immunosuppression. The administration may be effected at once or repeatedly over a period of several days or months.

Anti-histone H1 antibodies have been described above in detail for immunosuppressants of the invention, but the invention is not limited to these antibodies; antagonists exhibiting reactivity for the proteins (especially antigenic proteins) of the invention are also expected to have similar immunosuppressing activity. This invention therefore also encompasses such antagonists containing low molecular weight compounds.

The immunosuppressants of the invention are particularly useful as transplant immunosuppressants, but they may also be effective for autoimmune diseases such as rheumatism, atopy or systemic lupus erythematosus.

### EXAMPLES

The invention will now be described in further detail based on the following examples; however, the invention should not be limited to these examples.

### EXAMPLE 1

Inducement of Rat Immunosuppressing Substances

Orthotopic liver transplant surgery was conducted with rats according to the procedure of Kamada et al. (cited above). Male PVG and DA rats weighing 200-300 g (Harlan) were used, and the DA rat livers were transplanted into the PVG rats. Blood was sampled periodically after the liver transplantation (day 7, 14, 18, 21 and 63). The blood was centrifuged at 100×g, the supernatants were collected, and the post-liver transplantation sera were obtained.

### EXAMPLE 2

MLR Assay of Rat Post-Liver Transplantation Sera

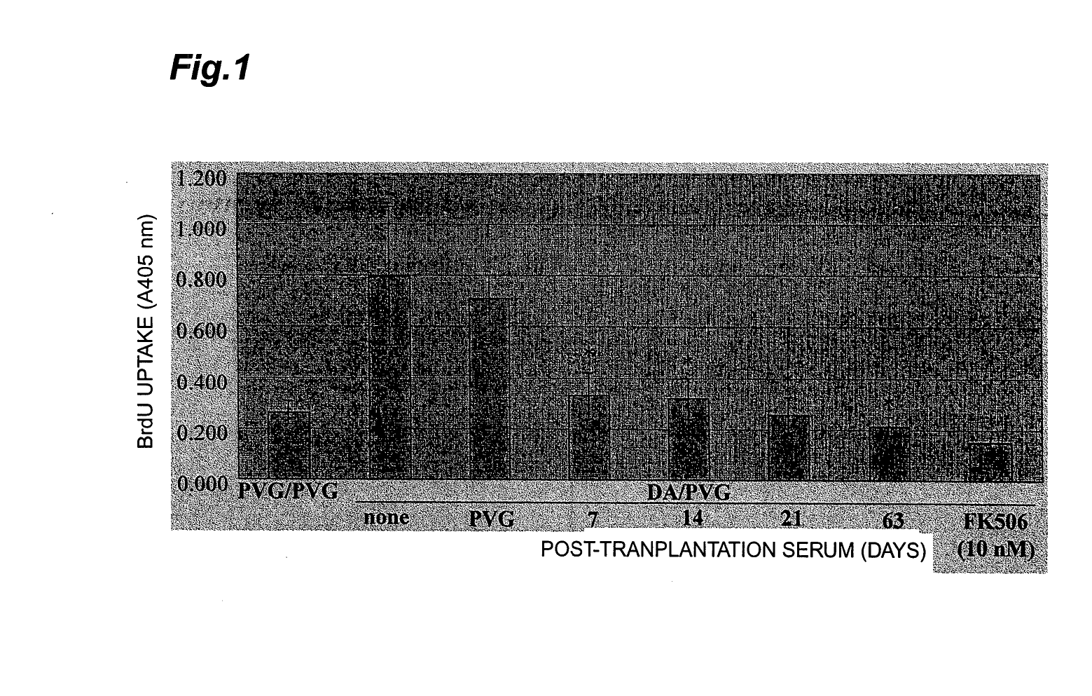

There were used untreated spleen lymphocytes of a PVG rat (responder cells) and mitomycin C (Wako Pure Chemical Industries Co., Ltd.)-treated spleen lymphocytes derived from a DA rat (stimulator cells). The responder cells and stimulator cells were prepared to 1×10⁶ cells/ml and 8×10⁶ cells/ml respectively, in growth medium (RPMI-1640 (GIBCO BRL), fetal bovine serum (LifeTech Oriental Co., Ltd.)). After seeding 100 μl of each cell suspension in a 96-well round-bottom plate (Nunc Brand Products), 4 μl of PVG control serum or the post-liver transplantation serum obtained in Example 1 (final concentration: approximate 2%) was added and culturing was performed for 5 days. As a positive control, culturing was performed with addition of tacrolimus (FK506: Fujisawa Pharmaceutical Co., Ltd.). A BrdU labeling and detection kit III (Roche Diagnostics) was used to measure the rate of cell growth based on the amount of bromodeoxyuridine (BrdU) taken up into the intracellular DNA by the 5th day of culturing. A greater amount of uptake indicates a greater level of cell proliferation (T cell activation). The results are shown in FIG. 1. FIG. 1 shows immunosuppressing activity in the serum sampled after liver transplantation; the immunosuppressing activity of the serum from the intermediate stage to the later stage post-transplantation was equivalent to that of the potent immunosuppressant tacrolimus. It was thus demonstrated that an immunosuppressing factor is present in post-liver transplantation recipient PVG serum.

### EXAMPLE 3

Analysis of Rat Post-Liver Transplantation Sera

The rat post-liver transplantation sera were analyzed by Western blotting. After placing 1 μl each of DA and PVG control sera and rat post-liver transplantation serum (Example 1) in a 1.5 ml tube, 3 μl of SDS-PAGE sample buffer (125 mM Tris-HCl, pH 6.8, 10% (v/v) 2-mercaptoethanol, 4% (w/v) SDS, 20% (v/v) glycerol, 0.01% (w/v) bromophenol: Wako Pure Chemical Industries Co., Ltd.) was added, and the mixture was boiled for 5 minutes to prepare samples. A 1 μl portion of each sample solution was applied to 10% polyacrylamide gel and subjected to electrophoresis.

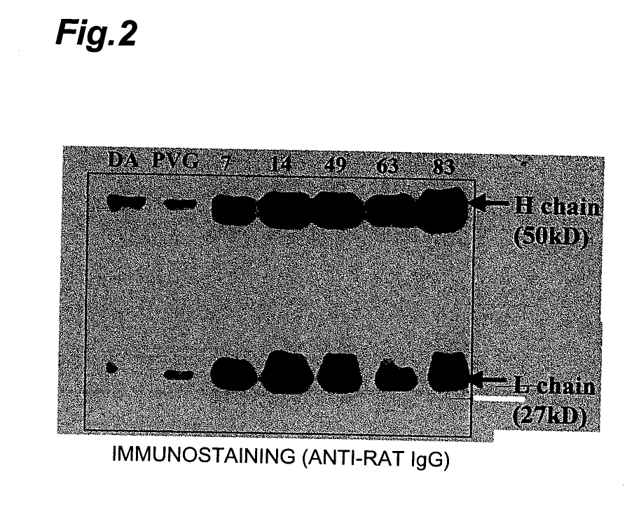

After electrophoresis, a semi-dry blotting apparatus (ATTO Corporation) was used for blotting (blotting buffer: 25 mM Tris-HCl, 192 mM glycine, 0.1% SDS, 20% methanol, Wako Pure Chemical Industries Co., Ltd.) on a 0.45 μm polyvinylidene fluoride membrane (PVDF membrane, Immobilon-P Transfer Membrane, Millipore). The blotted PVDF membrane was immersed in a blocking solution (3% skim milk, 0.1% polyoxyethylene(20) sorbitan monolaurate (Tween20), 10 mM phosphate buffer, Wako Pure Chemical Industries Co., Ltd.), and shaken at room temperature for 1 hour. The PVDF membrane was immersed in PBST (0.1% Tween20, 10 mM phosphate buffer) containing a 1/50,000 (v/v) volume of goat anti-rat IgG antibody for the H and L chains of rat IgG (anti-rat IgG) and HRP (Biosource International), and shaken at room temperature for 1 hour. The PVDF membrane was then rinsed in PBST once for 15 minutes and 3 times for 5 minutes, provided for detection with an ECL Western Blotting Detection Set (Amersham-Pharmacia Biotech), and exposed onto X-ray film (Fuji Photo Film Co., Ltd.) which was then developed. The results are shown in FIG. 2. In this figure, the top spot row corresponds to the H chain (50 kD), and the bottom spot row corresponds to the L chain (27 kD).

The results shown in FIG. 2 indicate that the IgG component immunostained in the post-liver transplantation serum increased with time compared to the PVG control.

### EXAMPLE 4

Separation of IgG Fractions from Rat Post-Liver Transplantation Sera

Rat post-liver transplantation sera (14th and 63rd days) were fractionated using an FPLC system (Amersham-Pharmacia Biotech) and Hitrap Protein G (Amersham-Pharmacia Biotech). A Hitrap Protein G column was equilibrated with 10 ml of 10 mM phosphate buffer (pH 7.2), and then 1 ml of the rat post-liver transplantation serum diluted 10-fold with 10 mM phosphate buffer (pH 7.2) was applied. After rinsing the column with 10 mM phosphate buffer (pH 7.2), the adsorbed fraction (IgG) was eluted with 10 ml of 0.1 M glycine buffer (pH 2.5, Wako Pure Chemical Industries Co., Ltd.). The pH of the solution was adjusted to neutral by addition of 1 M Tris-HCl, pH 9.0. The eluted fraction was concentrated using a 0.22 μm filter (Millipore), and the buffer was replaced with phosphate buffer to the original serum volume (100 μl).

### EXAMPLE 5

Immunosuppressing Activity of IgG Fraction in Rat Post-Liver Transplantation Serum

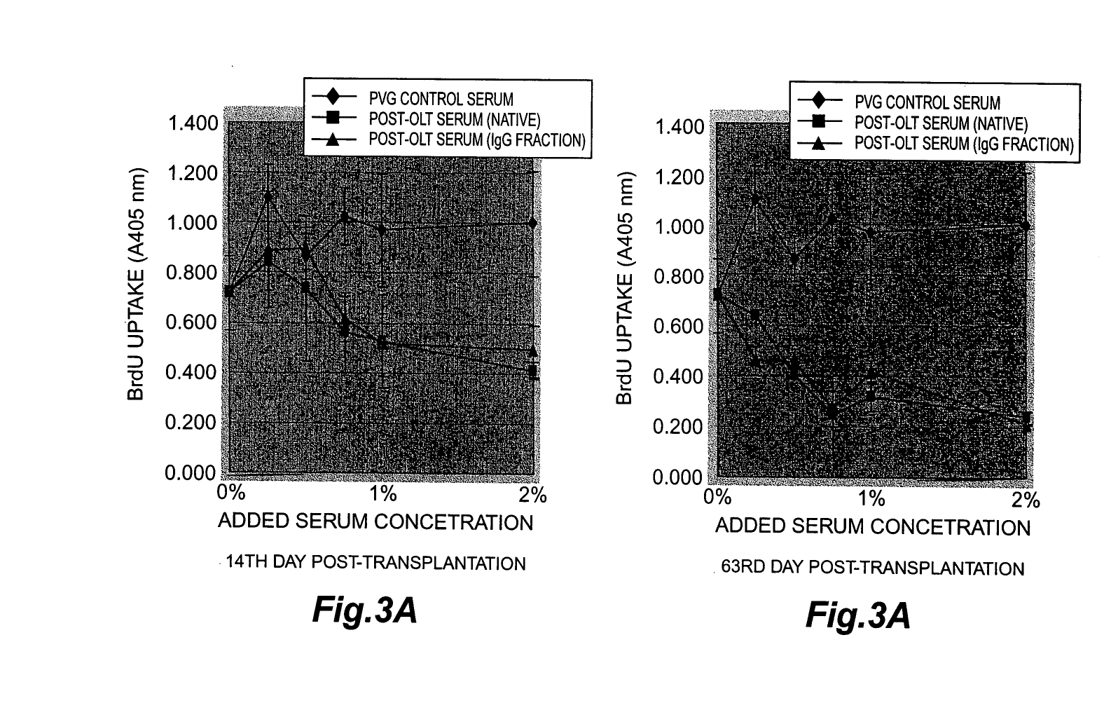

The immunosuppressing activities of the IgG fractions obtained in Example 4 were measured by MLR assay in the same manner as Example 2. That is, the immunosuppressing activity of each of the PVG control serum, the 14th day post-liver transplantation serum (native), its IgG fraction, the 63rd day post-liver transplantation serum and its IgG fraction were assayed after adjustment of the respective concentrations. The results are shown in FIG. 3. As FIG. 3 clearly shows, both IgG fractions exhibited approximately the same immunosuppressing activity as the native serum. These data indicate that the immunoactive substance in the rat post-liver transplantation serum is the IgG fraction.

### EXAMPLE 6

Antigens Recognized by Rat Post-Liver Transplantation Serum

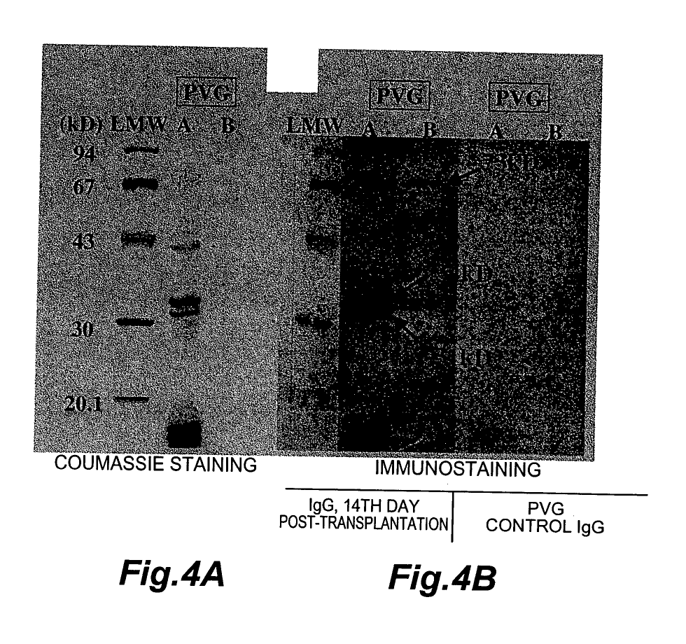

Spleen cells were obtained from a spleen extracted from a PVG rat. The spleen cells were mixed with 1% SDS solution in a proportion of 1:5 (v/v) and the mixture was thoroughly stirred. After adding 10 mg/ml phenylmethylsulfonyl fluoride (PMSF: Wako Pure Chemical Industries Co., Ltd.) in an amount of 1/30 of the total solution volume, the mixture was left on ice for 30 minutes to obtain a rat spleen cell extract. To 2.5 g 1 of the extract there was added an equivalent volume of non-reduced sample buffer (125 mM Tris-HCl, pH 6.8, 4% (w/v) SDS, 20% (v/v) glycerol, 0.01% (w/) bromophenol blue), and the solution was boiled for 5 minutes. A 5 μl portion of the sample solution was applied to 12.5% polyacrylamide gel for electrophoresis. After electrophoresis, it was blotted onto a PVDF membrane in the same manner as Example 3. After blocking, incubation was performed in a blocking solution containing a 1/5000 (v/v) volume of the post-liver transplantation serum (14th day). The PVDF membrane was rinsed in PBST once for 15 minutes and 3 times for 5 minutes, and then shaken for 1 hour in PBS containing a 1/50,000 (v/v) volume of rat IgG antibody. The PVDF membrane was again rinsed in PBST once for 15 minutes and 3 times for 5 minutes, and then provided for detection with an ECL Western Blotting Detection Set and exposed onto X-ray film which was developed. The results are shown in FIG. 4(b).

The electrophoresed gel was immersed in a staining solution (0.25% Coumassie Brilliant Blue R-250 (CBB)/ethanol:acetic acid:water=9:2:9, Wako Pure Chemical Industries Co., Ltd.) and stained while shaking for 1 hour. After staining, it was immersed in a decoloring solution (ethanol:acetic acid:water=25:8:65) for decoloration while stirring for 1 hour. This was followed by immersion in a preserving solution (methanol:acetic acid:water=10:15:175, Wako Pure Chemical Industries Co., Ltd.), for decoloration of the background. An LMW marker kit (Amersham-Pharmacia Biotech), was used as the protein molecular weight marker. The results are shown in FIG. 4(a).

Based on the results in FIG. 4(a) and FIG. 4(b), immunostaining of the post-liver transplantation serum indicated specific recognition of three different proteins (molecular weights: 73 kD, 34 kD, 31 kD) derived primarily from spleen cells.

### EXAMPLE 7

Immunosuppressing Activity of Immunodepleted Rat Post-Liver Transplantation Serum

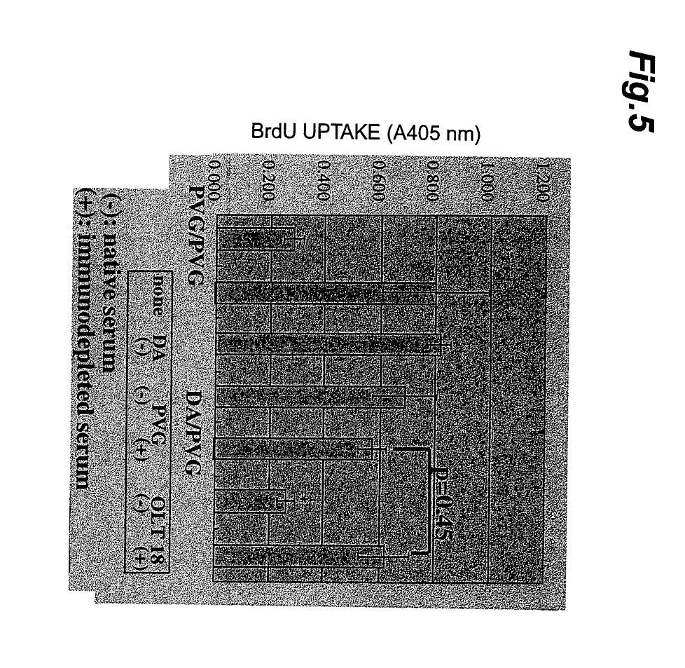

Spleen cells were obtained from a spleen extracted from a PVG rat. The spleen cells were ultrasonically treated and centrifuged, and the insoluble pellet was obtained. The rat post-liver transplantation serum (18th day) was added to the insoluble pellet in RPMI medium and incubated at 37° C. for 3 hours. After incubation, centrifugation was performed and the supernatant was added to an MLR assay culturing system for assay of the immunosuppressing activity in the same manner as Example 2. As controls there were used untreated post-liver transplantation serum (18th day), PVG serum (untreated and treated) and DA serum. The results are shown in FIG. 5.

The post-liver transplantation serum from which the spleen cell-derived antigenic protein-specific IgG component had been removed using the same protein had lower immunosuppressing activity compared to the native serum.

### EXAMPLE 8

Separation and Sequencing of Antigenic Proteins from Rat Spleen Cells

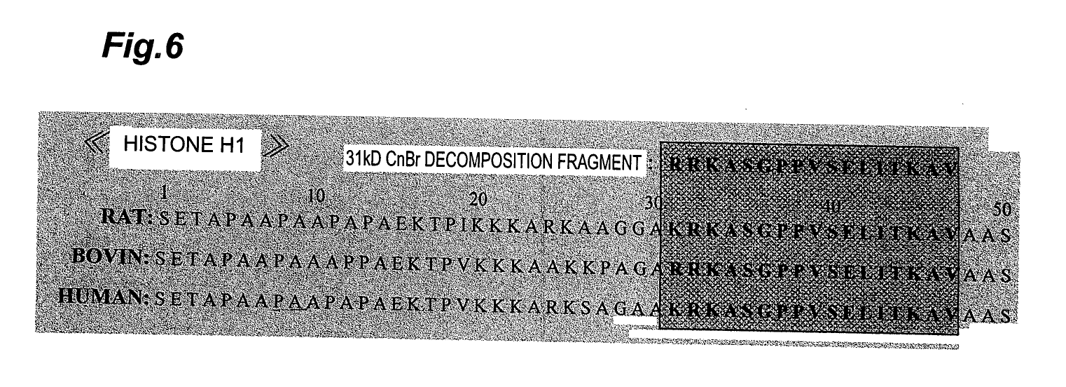

The 31 kD protein separated by the electrophoresis described in Example 6 was purified with a CNBr-activated Sepharose 4B gel (Amersham-Pharmacia Biotech) affinity column. The CNBr decomposition fragment of the 31 kD protein obtained in this manner was analyzed with a protein sequencer (476A Protein Sequencer, ABI). The determined partial sequence was used in a BLAST search to determine homology with known proteins, and partial matching was found with the sequence of human histone H1, among others. FIG. 6 shows the alignment of a partial sequence of human histone H1 against the aforementioned sequenced portion. The homologous portion (boxed section in the figure) is conserved in human, bovine and rat. The 31 kD protein was mixed with an isoelectric point measuring buffer to prepare a sample solution. The sample solution was then applied onto a swelled Immobiline Drystrip (Amersham-Pharmacia Biotech) by a conventional method and isoelectric point electrophoresis conducted. Staining of the gel after electrophoresis indicated a protein with an isoelectric point of 5.3.

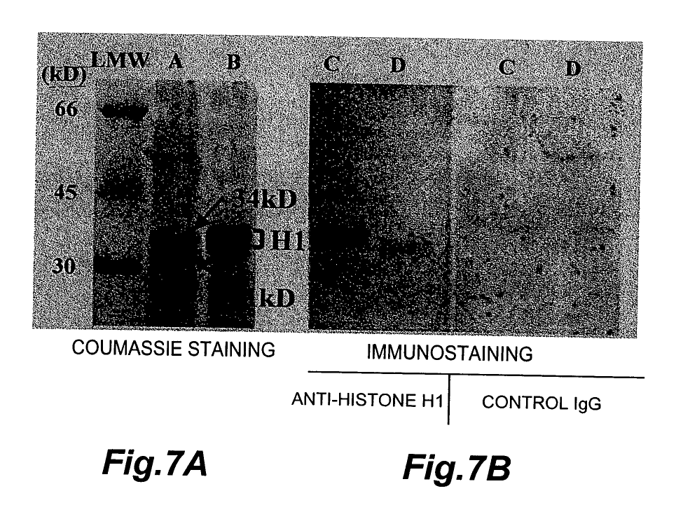

The electrophoresis was also followed by Coumassie staining in the same manner as described in Example 6. The results are shown in FIG. 7(a). In this figure, lane A represents the spleen cell extract, and lane B represents bovine histone H1.

The 31 kD protein and 34 kD protein separated and purified by electrophoresis were immunostained by Western blotting in the same manner as Example 3. The antibodies used for staining were anti-IgG antibody (control) (described below) and anti-histone H1 antibody (described below). The results of immunostaining are shown in FIG. 7(b). The results indicated that the approximately 31 kD and 34 kD proteins were recognized by the anti-histone H1 antibody. In the figure, lane C represents the purified 34 kD protein and lane D represents the purified 31 kD protein.

### EXAMPLE 9

Separation and Purification of Novel Proteins from Rat Post-Liver Transplantation Serum

The proteins were separated and purified according to the method of Oda et al. (cited above). A 5 ml portion of rat post-liver transplantation serum (63rd day) was applied to a DEAE Sepharose column equilibrated with 20 mM Tris-HCl (pH 7.4), and eluted with 20 mM Tris-HCl (pH 7.4) containing 0.5 M NaCl. The eluate was applied to a CNBr-activated Sepharose 4B gel (Amersham-Pharmacia Biotech) affinity column, and the protein fraction was eluted using 0.1 M glycine (pH 2.5). The protein fraction was separated to produce a 37 kD protein. The protein was mixed with an isoelectric point measuring buffer to prepare a sample solution. The sample solution was applied onto a swelled Immobiline Drystrip (Amersham-Pharmacia Biotech) by a conventional method for isoelectric point electrophoresis. Staining of the gel after electrophoresis indicated a mixture of 4 different proteins with isoelectric points of 4.86, 4.86, 5.04 and 5.04, respectively.

### EXAMPLE 10

In vitro Immunosuppressing Activity of Anti-Histone H1 Antibody

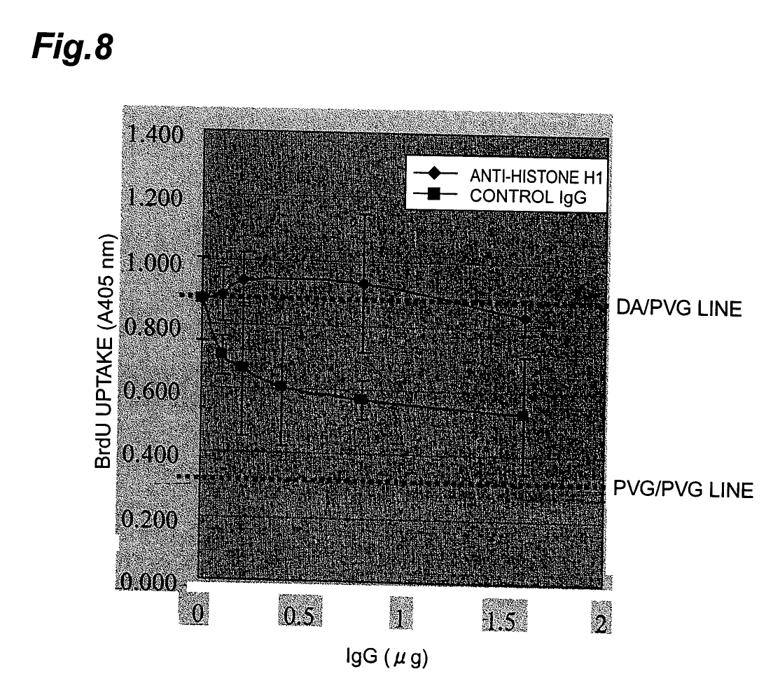

The aforementioned MLR assay was used to test the in vitro immunosuppressing activity of anti-histone H1 antibody. The anti-histone H1 antibody used was a commercially available anti-histone H1 rabbit polyclonal antibody (Santa Cruz Biotechnology, Catalog No. sc-10806). The antibody was polyclonal antibody against the full-length human histone H1. Rabbit IgG (Santa Cruz Biotechnology, Catalog No. sc-2027) was used as the control antibody. Before the assay, both antibodies were subjected to desalting treatment with an Ultrafree MC5000NMWL Filter Unit (Millipore), and the buffer was replaced with PBS. Each antibody pretreated in this manner was added to an MLR assay culturing medium in the manner described in Example 2 with 0-1.6 μg of IgG, and cultured. FIG. 8 shows the results of measuring the rate of cell proliferation in the same manner. As FIG. 8 clearly shows, the anti-histone H1 antibody possesses immunosuppressing activity.

### EXAMPLE 11

In vivo Immunosuppressing Activity of Anti-Histone H1 Antibody

The immunosuppressing activity of the anti-histone H1 antibody was tested by a heterotopic heart transplant technique which served as an organ transplant rejection model (Experimental liver transplantation 19:CRC Press, 1988). The test was conducted using a commercially available anti-histone H1 rabbit polyclonal antibody (Santa Cruz Biotechnology, Catalog No. sc-10806) as in Example 10. Following the procedure of Kamada et al. (cited above), DA rat (donor) hearts were transplanted into PVG rats (recipients), and 1 ml of anti-histone H1 antibody solution prepared to 300 μg/ml with physiological saline (Otsuka Pharmaceutical) was subcutaneously administered. An untreated group was used as a control group. The average number of days of successful take was <9.0 in the untreated group, while the average number of days was 14.3 in the anti-histone H1 antibody-administered group. The number of days of successful take of the transplanted heart grafts were, therefore, increased in the administered group. When the rats in the administered group were abdominally operated to observe the organs, no notable abnormalities were observed, as in the untreated group. This observation of immunosuppressing activity of the anti-histone H1 antibody in vivo therefore demonstrated a clear rejection-suppressing effect of the antibody in transplantation.

### EXAMPLE 12

Assay of Anti-Histone Antibody Titer in Rat Post-Liver Transplantation Serum

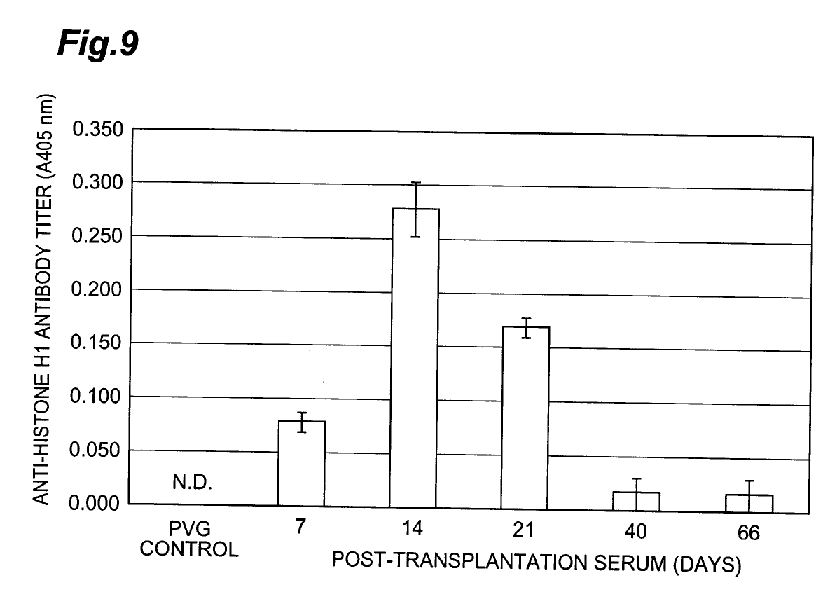

The anti-histone antibody titers of rat post-liver transplantation sera (7th to 66th day) were assayed by the standard ELISA method. The results are shown in FIG. 9. It was found that the antibody titer increased during 14-21 days after transplantation.

1. An immunosuppressant comprising an antibody against histone H1.

2. The immunosuppressant according to claim 1, wherein the antibody against histone H1 is an anti-histone H1 polyclonal antibody.

3. The immunosuppressant according to claim 1, wherein the histone H1 is human-derived.

4. The immunosuppressant according to any one of claims 1 to 3, characterized by being used for transplant immunosuppresion.
